(19) United States
(12) Patent Application Publication
(10) Pub. No.: US 2012/0035675 A1
(43) Pub. Date: Feb. 9, 2012

(54) EXTERNAL DEFIBRILLATOR WITH ADAPTIVE CPR DURATION

(75) Inventors: Robert G. Walker, Bothell, WA (US); Daniel W. Piraino, Seattle, WA (US); James M. Owen, Redmond, WA (US)

(73) Assignee: PHYSIO-CONTROL, INC., Remond, WA (US)

(21) Appl. No.: 13/272,960

(22) Filed: Oct. 13, 2011

Related U.S. Application Data
(62) Division of application No. 12/110,174, filed on Apr. 25, 2008.

(51) Int. Cl. A61N 1/39; A61H 31/00

(52) U.S. Cl. 607/3

(57) ABSTRACT

A method and apparatus for a defibrillating system is disclosed that monitors the patient during treatment and then uses the information it gathers to adjust treatment protocols during treatment based on the patient's response. The protocols may include adaptive rhythm analysis intervals, adaptive CPR intervals, and adaptive shock stacks. A method of operating a defibrillator may include the steps of: obtaining a data set on at least one physiological parameter of a patient in a first data gathering interval; performing an analysis of the data set; and determining a time interval between the analysis of the first data set and a second data set, or the duration of a CPR interval, or the number of shocks in a shock stack, based on the result of the analysis of the data set.

### CROSS-REFERENCE TO RELATED APPLICATIONS

This application is a division of U.S. patent application Ser. No. 12/110,174 entitled “EXTERNAL DEFIBRILLATOR WITH ADAPTIVE PROTOCOLS” filed Apr. 25, 2008, currently pending.

### TECHNICAL FIELD

This invention relates generally to external defibrillators, and more specifically to AEDs and protocols which they carry out.

### BACKGROUND

A cardiac arrest is a life-threatening medical condition in which a person's heart fails to provide enough blood flow to support life. During a cardiac arrest, the electrical activity may be disorganized (ventricular fibrillation), too rapid (ventricular tachycardia), absent (asystole), or organized at a normal or slow heart rate (pulseless electrical activity). A person treating a cardiac arrest victim may apply a defibrillation shock to the patient in ventricular fibrillation (VF) or ventricular tachycardia (VT) to stop the unsynchronized or rapid electrical activity and allow a perfusing rhythm to commence. External defibrillation, in particular, is provided by applying a strong electric pulse to the patient's heart through electrodes placed on the surface of the patient's body. The brief pulse of electrical current (commonly referred to as a shock) is provided to halt the fibrillation, giving the heart a chance to start beating with a more normal rhythm. If a patient lacks a detectable pulse but has an ECG rhythm of asystole or pulseless electrical activity (PEA), an appropriate therapy includes cardiopulmonary resuscitation (CPR), which causes some blood flow.

The probability of surviving a cardiac arrest depends on the speed with which appropriate medical care is provided to a patient experiencing the cardiac arrest. To decrease the time until appropriate medical care is provided, it has been recognized that those persons who are first to arrive at the scene, “first responders,” should be provided with an automated external defibrillator (AED). Typically the AED is a small, portable device that analyzes the heart's rhythm and delivers a defibrillation shock if it determines that the heart is in a condition where such a shock is an appropriate therapy.

AEDs are generally designed for use by the first responder, who may be an emergency medical services worker, a firefighter, a police officer, or a layperson with minimal or no training on the use of an AED. AEDs which guide the first responder through each step of the defibrillation process by providing instructions in the form of aural voice prompts and/or visual prompts are commercially available.

Protocols have been developed for AEDs that typically dictate the sequence and timing of activities that the AED moves through in guiding and delivering care to a cardiac arrest patient. The protocols typically include particular numbers of successive cycles of ECG analysis and shock delivery (sometimes called “shock stacks”), intervals where the responder is to perform CPR, analyses of the patient's ECG (sometimes referred to as “rhythm analysis”), potential triggers for rhythm analyses, and visual and/or audio prompts associated with these activities. AEDs typically employ fixed and predetermined protocols to all patients. These protocols are based upon the American Heart Association Guidelines recommendations, and allow some flexibility in the initial configuration of an AED's protocols (for example, in duration of CPR intervals). However, once configured, the typical AED will essentially repeat the same protocol within and among all patients it treats.

### BRIEF SUMMARY

In an embodiment, a method of operating a defibrillator may include the steps of: obtaining a first data set on at least one physiological parameter of a patient in a first data gathering interval; performing an analysis of the first data set; obtaining a second data set on a physiological parameter of the patient in a second data gathering interval subsequent to the first analysis; performing an analysis of the second data set; and determining a time interval between the analysis of the first data set and the analysis of the second data set based on the result of the analysis of the first data set.

The step of performing the analysis of the first data set may include the step of determining whether the patient's heart is in ventricular fibrillation (VF); and the step of determining a time interval may include the step of selecting a first time interval if VF is present and a second time interval if VF is not present, the first time interval being shorter than the second time interval.

The first data set may be the initial data set collected when the defibrillator is initially attached to the patient.

The method may further include obtaining a third data set on a physiological parameter of the patient in a second data gathering interval subsequent to the second analysis; performing an analysis of the third data set; and determining a time interval between the analysis of the second data set and the analysis of the third data set based on the result of the analysis of the second data set.

The method may further include obtaining a third data set on a physiological parameter of the patient in a second data gathering interval subsequent to the second analysis; performing an analysis of the third data set; and determining a time interval between the analysis of the second data set and the analysis of the third data set based on the result of the analysis of the second data set. The step of determining time intervals may include selecting a first time interval length if VF is present and a second time interval length if VF is not present, the first time interval length being shorter than the second time interval length.

The method may further include delivering defibrillating electrical therapy if VF is present.

The at least one sensed physiological parameter may be selected from among ECG, patient impedance, heart rhythm, heart rate, cardiac output, blood flow, level of perfusion, patient temperature, and respiration rate.

Adjusting the time interval may further include performing an analysis of VF characteristics and adjusting the time interval duration in response to the result of the VF characteristics analysis.

In another embodiment, a method of operating a defibrillator may include the steps of: obtaining a data set on at least one sensed physiological parameter of a patient; performing an analysis on the data set; determining a duration of a subsequent time interval during which CPR is administered based on the result of the analysis of the data set. The step of performing the analysis of the data set may include the step of determining whether the patient's heart is in ventricular fibrillation (VF); and the step of determining the duration of the CPR interval may include the step of selecting a first CPR duration if VF is present and a second CPR duration if VF is not present, the first CPR duration being shorter than the second CPR duration.

The data set may be the initial data set collected when the defibrillator is attached to the patient.

The step of determining CPR durations may include selecting a first CPR duration length if VF is present and a second CPR duration length if VF is not present, the first CPR interval duration length being shorter than the second CPR interval duration length.

The method may further include instructing delivery of a defibrillating shock if the patient is in VF.

The step of adjusting the duration of CPR intervals may further include: performing an analysis of VF characteristics and adjusting the duration of CPR intervals in response to the result of the VF characteristics analysis.

The method may further include instructing delivery of an immediate defibrillation shock if the result of the VF characteristics analysis meets a predetermined criterion.

The sensed physiological parameter includes at least one parameter selected from among ECG, patient impedance, heart rhythm, heart rate, cardiac output, blood flow, level of perfusion, patient temperature, and respiration rate.

In another embodiment, a method of operating a defibrillator may include obtaining a first data set on at least one physiological parameter of a patient; performing an analysis of the first data set; and determining a number of defibrillator shocks in a shock stack based upon the result of the analysis of the first data set.

This method may further include the steps of: delivering the shock stack to the patient; prior to the step of delivering the shock stack, obtaining a second data set and analyzing the second data set.

This method may further include the steps of: delivering the shock stack to the patient; after the step of delivering the shock stack, obtaining a second data set on at least one physiological parameter of a patient; performing an analysis of the second data set; and determining a number of defibrillation shocks in at least one subsequent shock stack based upon the result of the analysis of the second data set.

The method may further include the steps of delivering the shock stack to the patient; after the delivery of shock in the shock stack, obtaining a post-shock data set on a physiological parameter of a patient and performing an analysis of the post-shock data set; and determining a number of defibrillator shocks in at least one subsequent shock stack based upon the result of the analysis of at least one post-shock additional data set. The determination of defibrillator shocks in the at least one subsequent shock stack may be further based on the analysis of the first data set, or on the analysis of the at least one additional data sets.

The sensed physiological parameter may be at least one parameter selected from among ECG, patient impedance, heart rhythm, heart rate, cardiac output, blood flow, level of perfusion, patient temperature, and respiration rate.

An embodiment of the invention may provide a defibrillator capable of adjusting its treatment protocols during treatment based on ECG rhythm analysis of the patient. Furthermore, other desirable features and characteristics of the present invention will become apparent from the subsequent detailed description and the appended claims, taken in conjunction with the accompanying drawings and the foregoing technical field and background.

### BRIEF DESCRIPTION OF THE DRAWINGS

FIG. 1 is a schematic view of a defibrillation system connected to a patient in accordance with an exemplary embodiment of the invention;

FIG. 2 is a block diagram of the external defibrillator of FIG. 1;

FIG. 3 is a flowchart that illustrates a method of operating the defibrillator of FIGS. 1 and 2 according to a first embodiment of the invention;

FIG. 4 is a flowchart that illustrates another method of operating the defibrillator of FIGS. 1 and 2 according to a second embodiment of the invention; and

FIG. 5 is a flowchart that illustrates another method of operating the defibrillator of FIGS. 1 and 2 according to a third embodiment of the invention.

FIG. 6 is a timeline (not to scale) showing an example of a sequence of analyses and defibrillation shocks according to another embodiment.

### DETAILED DESCRIPTION

The following detailed description is illustrative in nature and is not intended to limit the invention or the application and uses of the invention. Furthermore, there is no intention to be bound by any expressed or implied theory presented in the preceding technical field, background, brief summary or the following detailed description.

The invention may be described herein in terms of functional and/or logical block components and various processing steps. It should be appreciated that such block components may be realized by any of a number of hardware, software, and/or firmware components configured to perform the specified functions. For example, an embodiment of the invention may employ various integrated circuit components, e.g., memory elements, processing elements, logic elements, look-up tables, or the like, which may carry out a variety of functions under the control of one or more microprocessors or other control devices. In addition, those skilled in the art will appreciate that the present invention may be practiced in conjunction with any of a number of data transmission protocols and that the system described herein is merely an illustrative application for embodiments of the invention.

For the sake of brevity, conventional techniques related to defibrillator devices, automated external defibrillators (AED), related control signal processing, data transmission, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in a practical embodiment.

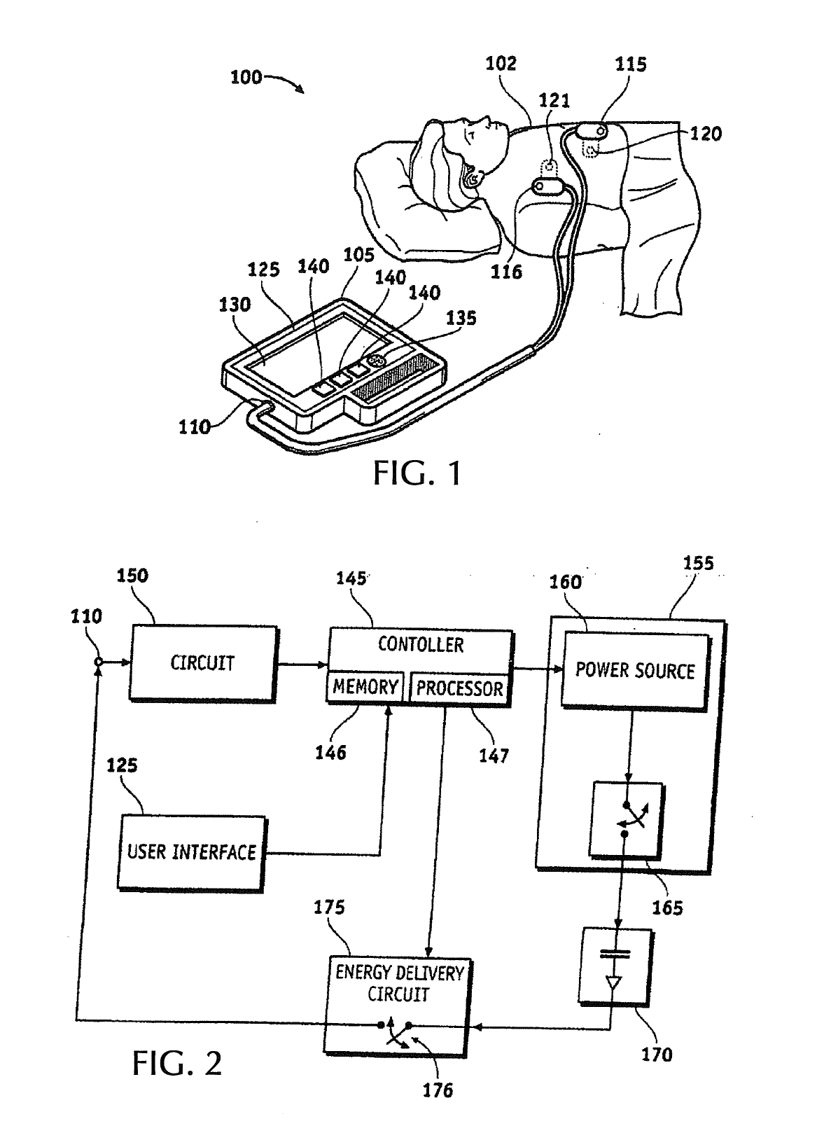

FIG. 1 shows an example of a defibrillating system 100 configured to be able to deliver a defibrillation shock to a cardiac arrest patient 102, such as a victim of ventricular fibrillation (VF). The illustrated defibrillating system 100 includes a defibrillator 105 having a connection port 110 that is configured to electrically connect defibrillator 105 to a pair of electrodes 115, 116. The defibrillator 105 may be any of a number of external defibrillators in accordance with the illustrated embodiment. For example, the defibrillator 105 can be an Automated or Automatic External Defibrillator (AED) or a manual monitor/defibrillator. While many of the exemplary embodiments of the invention apply to all types of external defibrillators, some of the embodiments are only for specific types, such as embodiments only for automated defibrillators or only for manual monitor/defibrillators.

Monitoring the patient may be accomplished by the defibrillating system 100 on any one or combination of a number of different physiological parameters. The defibrillator 105 preferably includes a user interface 125 having a display 130 that is configured to visually present to the user various measured or calculated parameters associated with the patient 102 and/or other information to a user of the defibrillator 105. For example, the display 130 can be configured to visually present electrocardiogram (ECG) and/or other physiological signals indicating the physical status of the patient 102, or instructions and/or commands, including prompts to perform cardiopulmonary resuscitation (CPR) therapy or other treatment instructions, to the user. As used in this document, CPR includes chest compressions, with or without ventilations. The display 130 can also be configured to present visual alerts, flashing lights or warnings to the user. The user interface 125 may also include an audio system 135 that provides an audio signal to aurally communicate voice prompts that deliver instructions or commands, monotonal, ascending, descending or quickening tones to indicate alerts or warnings, or any other suitable audio signals for communicating with the user. The user interface 125 may also include one or more input devices (such as, for example, switches, dials or buttons) 140 that are configured to receive commands or information from the operator. Additionally, the visual display 130 and audio system 135 may be configured to cooperate with one another.

The defibrillator 105 is configured to generate a charge that is delivered to the patient 102 as a defibrillation shock with one or more electrodes 115, 116. The one or more electrodes 115, 116 may also be configured to sense one or more physiological and/or physical parameters of the patient 102 and supply signals representative of these parameters to the defibrillator 105. The one or more physiological and/or physical parameters of the patient 102 may include information about the patient's heart using an electrocardiogram (ECG) signal obtained by one or more sensors attached to the chest, optionally as part of the defibrillation electrodes, continuous high frequency impedance signal, or a plethysmographic waveform used by pulse oximeters. The sensed physical parameters may include ECG data, heart rhythm data, heart rate data, cardiac output data, blood flow data, a patient's level of perfusion, respiration data, patient impendence, patient temperature and/or any other physical parameter that is used in the art to assess the physical condition of the patient 102. As shown in phantom in FIG. 1, the defibrillator 105 may additionally include one or more sensing electrodes 120, 121 to sense the physiological and/or physical parameters. In either configuration, the signals provided by the electrodes 115, 116 and/or one or more sensing electrodes 120, 121 are preferably used by the defibrillator 105 to evaluate and determine, among other things, selection of an appropriate treatment protocol or adjustment of the current treatment protocol. It may also determine whether a defibrillation shock should be applied to the patient 102 in accordance with techniques known to those of ordinary skill in the art. The defibrillator 105 may also evaluate the signals provided by the electrodes 115,116 and/or sensing electrodes 120, 121 to determine the waveform parameters (e.g., voltage, current, energy and/or duration), as well as magnitude and duration of the defibrillation shock.

FIG. 2 shows a simplified block diagram of an embodiment of circuitry that may be utilized by the defibrillator 105. The defibrillator 105 preferably includes a controller 145 coupled to the user interface 125 (e.g., switches or buttons 140 and/or display 130 as shown in FIG. 1), a circuit 150, a charging mechanism 155 that may include a power source 160 and a switch 165 to couple the power source 160 to the one or more energy storage devices (e.g., capacitors) 170 and an energy delivery circuit 175, which is illustrated as a switch 176 that is configured to selectively couple the one or more energy storage devices 170 to the connection port 110 under the control of the controller 145. The energy delivery circuit 175 may be implemented with any number of circuit configurations. The controller 145 may be a single processing unit or multiple processing units and may be implemented with software, hardware, firmware, or any combination thereof. The controller 145 is configured to at least partially control the operation of the defibrillator 105, including control of charging the one or more energy storage devices 170. It will be appreciated that the circuitry depicted in FIG. 2 is merely exemplary of a particular architecture, and that numerous other circuit architectures may be used to implement the operation of the defibrillator 105.

The controller 145 may include, among other things, a memory unit 146 and a processor 147. The controller 145 is configured to automatically update and/or continuously sense the sensed physical parameter(s) and adjust the treatment protocol accordingly in a manner described more fully below. The processor 147 may be any one of numerous known general purpose processors or an application specific processor that operates in response to program instructions, which may be stored in any of various forms of memory storage. It will also be appreciated that the controller 145 may be implemented using various other circuits, not just a programmable processor. The memory unit 146 is in operable communication with processing unit 147.

The processor 147 of the illustrated embodiments modifies the treatment protocol which the defibrillator will follow, based in part or wholly on the sensed physiological parameter(s). The modification may be accomplished by choosing from among two or more protocols or protocol segments based on the sensed parameter(s). Alternatively, the modification may be accomplished by the controller adapting one or more parameters of a particular protocol based on the sensed parameter. The adapted protocol parameter may include, for example, the time duration of an interval within a protocol or the number of times a recursive routine within a protocol is performed. Examples will be discussed in detail below. The memory unit 146 may contain the operating system, software routines and a plurality of protocols which the controller may choose among. For instance, the protocols may include protocols having various durations for ECG rhythm analysis intervals and CPR intervals, and various configurations of recursive shock and ECG analysis routines that may be changed during treatment based on sensed physiological or physical signals of the patient. The protocols may also include predetermined parameters based on such things as CPR therapy, the administration of oxygen therapy, drug therapy, or, checking the patient for a pulse or for normal breathing, monitoring Sa02, monitoring end tidal CO2, or blood pressure levels, or any other non-electric treatment known in the art that is appropriately administered to a patient with an arrhythmic heart condition. The memory 146 can also receive and store the patient's sensed physical parameters and can store historical data, lengths of time and rate of CPR treatments and defibrillation shocks previously discharged to the patient It will be appreciated that above-mentioned circuitry of the defibrillator 105 is merely exemplary of one method for storing operating software, software routines, and adaptive protocols, and that various other storage schemes may be implemented. It will be appreciated that the memory unit 146 could be integrally formed as part of the controller 145 and/or processing unit 147, or could be part of a device or system that is physically separate from the external defibrillator 105.

Many patients in cardiac arrest who are attached to an external defibrillator never experience a shockable rhythm. Some patients who do experience a shockable rhythm do not return to a shockable rhythm once fibrillation is terminated by a defibrillation shock. If a rescuer who had been delivering CPR is instructed to not touch the patient during rhythm analysis, this results in an interruption in the delivery of CPR. But a patient in a cardiac emergency who is not in a shockable rhythm may benefit from delivery of chest compressions and CPR therapy with as few interruptions as possible. In an embodiment of the invention, a protocol for initiating ECG analysis may be adaptable to the relatively low expectation of finding a shockable rhythm in patients in certain patients (such as those described above), and the process and prompts issued by the defibrillator may be adapted so as to maximize the time spent delivering CPR to the patient. The embodiments described below provide external defibrillators and methods of operating external defibrillators in which interruptions to CPR can be significantly decreased and time spent delivering CPR can be maximized.

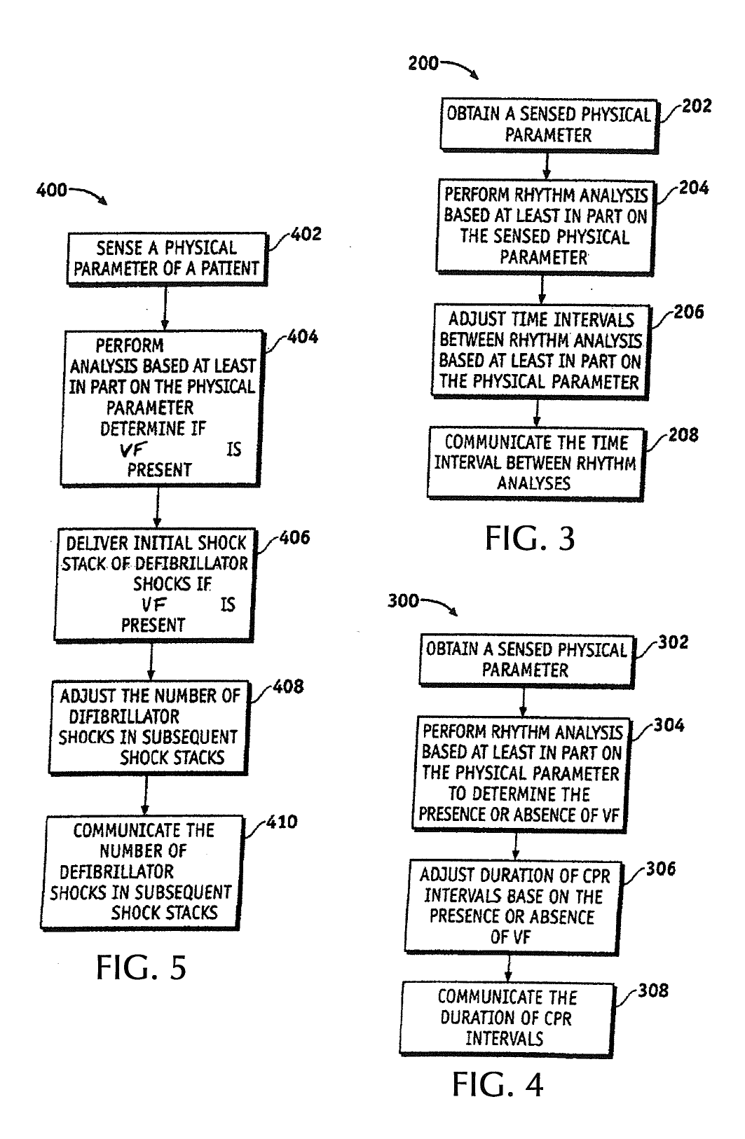

FIG. 3 is a flowchart that illustrates a method 200 of operating the defibrillator of FIGS. 1 and 2 according to an embodiment of the invention. In this embodiment, the time interval between successive rhythm analyses is adaptable based on a sensed physiological parameter(s) of the patient. In the illustrated method 200, if a rhythm analysis of data in a first time interval shows that VF is not present (resulting in a “no shock advised” decision), then a longer time interval between the first and the second successive rhythm analysis is chosen than would be the case if VF had been detected. If successive rhythm analyses instead result in “shock advised’ decision, i.e., if analysis shows that VF is present, then the time interval between the rhythm analyses can be held constant or can be shortened. An example of such an implementation would be where a predetermined number of seconds (for example, 30 seconds) is added to the time interval after each successive “no shock advised” decision. If a “shock advised” decision appears after one or more “no shock advised” decisions, the time interval between the rhythm analyses could either be held constant (in this example, no addition of 30 seconds), or revert or be reset to equal the initial time interval duration between the rhythm analyses.

The method 200 illustrated in FIG. 3 begins with the defibrillator obtaining a first data set on a sensed physiological parameter of a patient in a step 202. The first data set need not be the initial data set, as explained below. A data set may be all or a portion of the data obtained during a single data gathering interval or during more than one data gathering interval. The sensed physiological parameter in the illustrated embodiment is ECG data, but other parameters that may be used to assess a patient's condition, including for example, heart rate data, cardiac output data, blood flow data, a patient's level of perfusion, or respiration data, may be used in alternate embodiments. In the embodiment illustrated in FIG. 3, the defibrillator then performs an ECG analysis using the sensed physiological parameter information in a step 204. The time interval between analyses is then adjusted based at least in part on the physiological parameter in step 206. The defibrillator may then communicate the time interval between analyses (step 208), either displaying the information on the display or audibly through the audio system.

The time interval which is adjusted may be between analysis of an initial data set (i.e., the initial data set obtained from a patient when electrodes from the defibrillator are first applied to the patient) and the immediately subsequent analysis of a second data set, or it may be the time interval from any analysis to the next subsequent analysis. Or, the time interval may be between any analysis and any other future analysis. For example, if an analysis is done at time T0 and other analyses are done at T1, T2, and T3, then the time interval between any pair may be adjusted (e.g., between T0 and T1, or T0 and T3, or T1 and T3). Adjusting a time interval may entail determining a time duration of an interval between analyses. There may be a default interval duration between a given pair of analyses which may be lengthened or shortened depending on the outcome of the first analysis of the pair.

The determination of a time interval between analyses may be a determination to deviate (or not) from a default time interval or a predetermined standard for such time interval, or from a previous time interval duration, or it may be an independent determination of time interval duration without reference to a default, standard or another time interval.

In addition to the variable time intervals between physiological parameter analyses that would result from the above-described process of adapting the rhythm analysis interval, there are other embodiments in which CPR treatment intervals may be adapted based on ECG or other patient parameter input. In a treatment scheme in which a rescuer is prompted to deliver CPR after the attachment of the defibrillator electrodes to the patient, the duration of the initial CPR interval may be determined based on the initial sensed ECG rhythm. Patients presenting in PEA/asystole only rarely develop VF. Thus, in one embodiment, the defibrillator would prompt for a longer duration of CPR (for example, 3 minutes of CPR) when the initial ECG rhythm is PEA/asystole (because defibrillation will likely not be needed at any point for such a patient), and prompt for a shorter duration of CPR (for example, 1 minute) when the initial rhythm is found to be VF.

In addition, the output of an analysis of VF characteristics which provides an indication of the likelihood that a defibrillating shock will be successful, such as the shock success predicative analysis techniques described in U.S. patent application Ser. No. 11/095,305 filed on Mar. 31, 2005, U.S. Patent Application Publication No. 2004/0220489, or U.S. Pat. No. 6,438,419, all of which are hereby incorporated by reference, may be used to adjust the duration of the initial CPR interval. Duration of CPR may be based on whether the outcome of a VF characteristics analysis meets a predetermined criterion. For example, if a shock success predicative analysis shows a likelihood of shock success to be lower than a threshold value, a relatively longer initial duration of CPR would be implemented. Where the analysis shows the initial rhythm is VF, with a likelihood of shock success higher than the given threshold, a shorter time interval for initial CPR would be implemented. In such a method, there would also be a shock success likelihood value above which immediate defibrillation with no prior CPR would be the implemented response.

In like manner, the duration of CPR intervals may be adapted depending on the elapsed time since the onset of the patient's condition. A defibrillating shock is less likely to be successful in defibrillating a patient if the patient has been in a VF condition for a longer period of time. In such a case, a longer period of CPR may be of greater benefit to the patient. The defibrillator may be equipped with a timer or an input mechanism through which information on elapsed times since the onset of the cardiac emergency can be supplied to the processor 147. The processor 147 can then adjust the duration of the initial CPR in response to the patient's down-time, i.e., the elapsed time since the onset of his condition, or since occurrence of another event, such as notification of emergency services, dispatch of emergency services or discovery of the unconscious patient. A system for a defibrillator to acquire and use data on elapsed time is described in U.S. Patent Application Publication No. US2006/012919, which is hereby incorporated by reference herein.

FIG. 4 is a flowchart that illustrates an example of a method 300 of operating the defibrillator of FIGS. 1 and 2 with adaptive CPR intervals. The method starts with the defibrillator obtaining a sensed physiological parameter, such as ECG, of a patient (step 302). An analysis is then performed by the defibrillator based on the initial sensed physiological parameter, (e.g., analysis of ECG) to determine the presence or absence of VF (step 304). The duration of successive CPR intervals is then adjusted based on the presence or absence of VF (step 306), the duration being longer if VF is present and shorter if VF is absent. The defibrillator may then communicate the duration of successive CPR intervals (step 308), either displaying the information on the display or audibly through the audio system. Duration of the prescribed CPR interval may be communicated as a time interval (for example, a prompt to “perform X seconds of CPR”), or by a prompt which guides the user to perform CPR for the prescribed time interval without explicitly stating the duration length (for example, “perform CPR until you hear the tone”), or a prompt to perform chest compressions and/or ventilations in pace with a metronome or other periodic prompting sounds, with the series of prompting sounds lasting for the prescribed time interval length.

The determination of a duration for a CPR interval may be a determination to deviate (or not) from a default duration or a predetermined standard for CPR duration or from the duration of a previous CPR interval, or it may be an independent determination of a duration without reference to a default, standard or another CPR interval.

Within a resuscitation protocol that has been implemented in external defibrillators, termination of the shockable rhythm is attempted by up to three defibrillation shocks delivered in close proximity to each other (i.e., without intervening CPR) in an arrangement known as a “shock stack”. In a typical three shock stack protocol, an ECG analysis is done to determine if the condition of the patient's heart is in a shockable rhythm. If it is, the first shock of the stack is delivered. An ECG analysis is then done to see if the heart is still in a shockable rhythm. If it is, the second shock is delivered; If it is not, the shock stack is terminated with only one shock of the three shock stack being delivered. Likewise, an ECG analysis is done after delivery of the second shock to see if the heart remains in a shockable rhythm, and if it is, the third shock of the stack is delivered. If the heart is no longer in a shockable rhythm after the second shock, the shock stack is terminated without delivery of the third shock of the stack. After delivery of the last shock in the stack (i.e., the third shock or the last shock delivered before termination of the stack), an ECG analysis is done to determine if the patient is in a shockable rhythm.

This type of treatment may not be optimal for some circumstances. In particular, the repetition of this type of shock stack in those patients where defibrillation repeatedly fails or where refibrillation rapidly ensues after each defibrillation shock is likely not optimal care. FIG. 5 is a flowchart that illustrates a method 400 of operating the defibrillator of FIGS. 1 and 2 with adaptive shock stacks in which the number of defibrillation shocks in a shock stack is adjusted based upon the accumulated ECG assessment of the response to all prior defibrillation shocks. The method 400 starts with obtaining a data set on one or more sensed physical parameter such as ECG of a patient (step 402). Other parameters which may be analyzed include patient impedance, heart rhythm, heart rate, cardiac output, blood flow, level of perfusion, patient temperature, and respiration rate.

In the illustrated embodiment, an analysis of the data set is then performed to determine if VF analysis present (step 404). An initial shock stack of defibrillation shocks is then delivered to the patient if VF is present (step 406). The number of shocks in this stack or in subsequent shock stacks may be adjusted based upon the results of the analysis. The number of shocks in a subsequent shock stack may be adjusted based on the patient's response to the prior shocks (step 408), as determined through analysis of physiological parameters. The number of shocks in any shock stack may be one or more shocks. In the illustrated embodiment, this response would be evidenced by analysis of the patient's ECG. The defibrillator may then communicate the number of defibrillation shocks to be delivered in a subsequent shock stack (step 410), either displaying the information on the display or audibly through the audio system.

The number of shocks in a shock stack may be adapted by obtaining and analyzing a first data set on at least one physiological parameter of a patient, and then determining a number of shocks in a shock stack based upon the result of the analysis of the first data set. The shock stack need not be the next immediate shock stack. For example, prior to delivery of the adjusted shock stack to the patient, a second data set may be obtained and analyzed. Successive shock stacks may be adjusted depending on the outcome of an analysis after delivery of each stack.

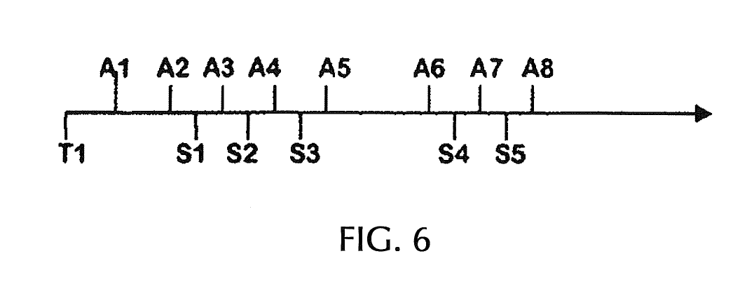

FIG. 6 shows a timeline with an example of a sequence of analyses and shocks using this method. The defibrillator is powered on with electrodes attached to the patient. Data collection begins at T1. An initial analysis of ECG is done at A1. In this example, the analysis at A1 shows the patient not to be in a shockable heart rhythm. For example, the patient may be in PEA or asystole at A1. After a time (during which a therapy other than defibrillating shock, such as CPR, may be administered) another ECG analysis is done at A2. In the illustration, the A2 analysis shows the patient to be in a shockable rhythm, so a shock stack composed of n shocks is delivered, where n can be 1, 2, 3 or more. In the illustration of FIG. 6, n=3 so a first, second and third shocks are delivered at times S1, S2 and S3 respectively. Each shock in the stack is followed by an ECG analysis, at A3, A4 and A5 respectively. In this example, had the analysis at either A3 or A4 shown that the patient was no longer in a shockable rhythm, the subsequent shocks in the stack would not have been delivered. Depending on the outcome of either or both of the analyses A1 or A2, the stack could have been chosen to include one, two, three or more shocks (with intervening analyses to confirm that delivery of the remaining shocks in the stack, if any, was appropriate patient care).

At some point after completion of the first stack, another analysis A6 is performed. If the analysis at time A6 shows the patient to be in a shockable rhythm, a second shock stack is delivered. In the illustrated example, the second stack includes only two shocks, S4 and S5 respectively, with interleaved analyses A7 and A8. This determination to deliver a two-shock stack could be based on any one of the analyses at times A1, A2, A3, A4, A5 or A6, or upon a combination of those analyses. The number n of shocks in any stack may be determined based on the initial analysis A1, or the analysis that was done after the last delivered shock in the immediately previous stack, or on any other one or more analyses. For example, a comparison of patient condition as indicated by comparing the outcome of analyses at times A1 and A3, for example, may provide information on how the patient responds to defibrillation shock which may make it desirable to include fewer or more shocks in a subsequent shock stack. Or, a finding in an initial analysis on a patient (for example, a finding of PEA or asystole at the analysis A1) may be used to determine n for al subsequent stacks.

The determination of a number of shocks in a stack may be a determination to deviate (or not) from a default number or a predetermined standard for shock number, or from the number of shocks in a previous shock stack or it may be an independent determination of how many shocks in a stack without reference to a default, standard or another shock stack.

In the preceding description, references to a “first” event are intended to designate temporal order of events (as compared to a “second” or subsequent event) and are not intended to be limited to events which are not preceded by any other event.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A defibrillator for defibrillating a patient, comprising:
a user interface;
a power source for storing a charge;
an energy storage device capable of receiving at least a portion of the charge, in which at least a fraction of the charge portion can be delivered from the energy storage device via electrodes as a shock so as to defibrillate the patient;
a user interface; and
a controller capable of:
obtaining a first data set on at least one sensed physiological parameter of the patient,
performing a first analysis on the first data set,
instructing delivery of at least one of the shocks if the result of the first analysis meets a predetermined criterion,
determining a first duration based on the first analysis, and
causing the user interface to instruct delivery of CPR for the first duration.

2. The defibrillator of claim 1, in which
the first analysis includes determining whether or not the patient's heart is in ventricular fibrillation (VF); and
the first duration is a first CPR duration if the patient's heart is in VF, and a second CPR duration different from the first CPR duration if the patient's heart is not in VF.

3. The defibrillator of claim 2, in which
the first CPR duration is shorter than the second CPR duration.

4. The defibrillator of claim 2, in which
if the patient's heart is in VF, VF characteristics are analyzed, and
the first CPR duration is adjusted in response to the result of analyzing the VF characteristics.

5. The defibrillator of claim 1, in which the controller is further capable of:
then obtaining a second data set on the sensed physiological parameter;
performing a second analysis on the second data set;
determining a second duration based on the second analysis, the second duration different than the first duration; and
causing the user interface to instruct delivery of CPR for the second duration.

6. The defibrillator of claim 1, in which
the sensed physiological parameter includes at least one parameter selected from the group consisting of: ECG, patient impedance, heart rhythm, heart rate, cardiac output, blood flow, level of perfusion, patient temperature, and respiration rate.
